(19) United States
(12) Patent Application Publication
(10) Pub. No.: US 2025/0382561 A1
(43) Pub. Date: Dec. 18, 2025

(54) Fermentation Device, System and Method for Producing Improved Fermentation Products

(71) Applicant: Edward Pei- Hong Lin, Canandaigua, NY (US)

(72) Inventor: Edward Pei- Hong Lin, Canandaigua, NY (US)

(21) Appl. No.: 18/878,259

(22) PCT Filed: Jun. 26, 2023
(86) PCT No.: PCT/US2023/026253

§ 371 Date: Dec. 23, 2024

Related U.S. Application Data
(60) Provisional application No. 63/356,016, filed on Jun. 27, 2022.

(51) Int. Cl. C12M 1/12; C12M 1/00; C12M 1/34; C12M 1/36

(52) U.S. Cl. CPC C12M 37/00; C12M 23/34; C12M 29/00; C12M 41/12; C12M 41/34; C12M 41/48

(57) ABSTRACT

A fermentation device, system and method for the production of improved fermentation products is disclosed. The device has a chamber, a temperature control element for heat transfer to/from the chamber, a ventilation fan, an air pump for delivery of oxygen into a fermentation bath, a humidifier, and an ultraviolet sterilization light source. The chamber may have partitions to allow for different fermentation processes to occur simultaneously. The system includes the fermentation device and a connection to a network such as a wi-fi network. The connection allows for not only control and programming of the device, but also collection and retrieval of various process parameters to improve upon the production of fermentation products.

### CROSS REFERENCE TO RELATED PATENT APPLICATIONS

This application claims priority to U.S. Patent Application Ser. No. 63/356,016 filed Jun. 27, 2022 entitled “Fermentation Device” by Edward Pei-Hong Lin, and to International Application Number PCT/US2023/026253 filed Jun. 26, 2023 entitled “Fermentation Device, System and Method for Producing Improved Fermentation Products”, the entire disclosures of which are incorporated herein by reference.

### TECHNICAL FIELD

The present invention relates generally to food processing, and more particularly to a device, system and associated methods for improved fermentation of a variety of foods and beverages in a safe and efficient manner.

### BACKGROUND ART

Fermentation is a biological process in which microorganisms, such as bacteria, yeast and mold, break down various substances into simpler components, often producing energy as well as other products such as alcohol and carbon dioxide. Fermentation processes occur naturally in all living organisms, including within our gastrointestinal tract, with a myriad of beneficial consequences. People have used fermentation processes to produce numerous food products for thousands of years, many of which were stumbled upon inadvertently. Some of the more commonly used fermentation processes include lactic acid fermentation, acetic acid fermentation, yeast fermentation and koji fermentation. Examples of commonly consumed foods that are produced through some sort of fermentation process include yogurt, fermented pickles, cultured butter, cultured cream, bread, kombucha, kefir, kimchi, miso, soy sauce, mirin, beer, wine and vinegar.

Fermentation products can greatly enhance the flavor of foods and have played an essential part in the evolution of all culinary arts. Some of these products, such as yogurt and soy sauce, are widely used and central to everyday recipes, such as in Middle Eastern and Asian cuisines. Many well-known chefs are increasingly using fermentation processes of various foods to enrich and add complexity of flavors and umami to the dishes that they serve in their restaurants.

In addition to the important role that fermentation has played in the kitchen, fermentation processes within the human body are now being recognized as playing a central role in human health. Scientific studies exploring how fermentation processes within our GI tract affect our health have advanced to the forefront of medical research over the last twenty years. Investigators are finding that the type and diversity of our gastrointestinal flora, which can number up to 100 trillion microorganisms, have significant but complex impacts on our health and have associations with a wide range of medical conditions, including obesity, diabetes, allergies, atherosclerosis, autoimmune diseases and cancer.

While fermented foods are often pleasing to gustatory sensations, they can also positively influence our gastrointestinal flora. Fermentation has, therefore, attracted a growing audience worldwide, particularly in the household. Many fermentation enthusiasts have created do-it-yourself fermentation chambers. Environmental conditions that are optimal for fermentation processes depend on the type of fermenting organism. Some fermentation processes require an environment without oxygenated air at a constant temperature, while other processes require a higher humidity and an environment with oxygen. Anaerobic fermentation processes can occur either in a vacuum capable container or bag, or in a water-containing fermentation bath. While water inherently contains oxygen, the oxygen concentration is significantly less in water (˜1%) than the oxygen concentration in the air (˜21% at sea level) in which we breathe. Because oxygen is much less available in water, anaerobic organisms are favored over aerobic organisms. The temperature at which fermentation occurs will affect the speed and efficacy of the fermentation processes.

Several types of kitchen-top appliances have been previously designed to maintain an enclosed chamber at programmed or manually set temperature. Examples of these types of devices include slow cookers, rice cookers, pressure cookers, and black garlic “fermenters”. Some of these devices could potentially be used to perform fermentation processes such as lactic acid fermentation. While these devices can all maintain the chamber at a set temperature for the purposes of cooking various types of dishes and grains, they are not able to perform fermentations that require conditions such as high humidity or oxygen, nor are they able to automate or even semi-automate a fermentation process.

Black garlic “fermenters” are devices that are essentially a chamber capable of maintaining a set temperature for long periods of time. While akin to fermentation, the process of making black garlic does not actually entail a fermentation process, but rather a chemical reaction, similar to caramelizing onions but with the process taking weeks to months. This chemical reaction, called a Maillard reaction, does not involve organisms, such as bacteria, yeast or mold, and is not a biological process like fermentation. As such, the term black garlic “fermenters” is actually a misnomer, as no fermentation process is actually taking place.

What is needed is a fermentation chamber that includes a variety of components such as an insulated chamber, a temperature control element(s), a temperature control and monitoring device, a humidifier with a humidity control and monitoring device, an air pump, a ventilation fan, and an ultraviolet light for surface sterilization. The present invention and the various embodiments described and envisioned herein address this heretofore unmet need.

### DISCLOSURE OF THE INVENTION

In accordance with the present invention, there is provided a Fermentation Device comprising a chamber, a temperature control element for heat transfer, a fan for ventilation of the chamber, an air pump for delivery of oxygen into a food to be fermented, a humidifier, and an ultraviolet sterilization light source.

The foregoing paragraph has been provided by way of introduction and is not intended to limit the scope of the invention as described herein.

### BRIEF DESCRIPTION OF THE DRAWINGS

The invention will be described by reference to the following drawings, in which like numerals refer to like elements, and in which:

FIG. 1 depicts a perspective view of a Fermentation Device of the present invention;

FIG. 2 depicts a front plan view of the Fermentation Device of FIG. 1;

FIG. 3 depicts a side plan view of the Fermentation Device of FIG. 1:

FIG. 4 depicts a rear plan view of the Fermentation Device of FIG. 1;

FIG. 5 depicts an alternate side plan view of the Fermentation Device of FIG. 1;

FIG. 6 depicts a top plan view of the Fermentation Device of FIG. 1;

FIG. 7 depicts a bottom plan view of the Fermentation Device of FIG. 1;

FIG. 8 depicts a perspective view of the Fermentation Device of FIG. 1 with the doors open and a removable partition depicted;

FIG. 9 depicts a perspective view of the Fermentation Device of FIG. 1 with the removable partition of FIG. 8 installed;

FIG. 10 depicts a perspective view of the Fermentation Device of FIG. 1 configured with trays;

FIG. 11 depicts a perspective view of the Fermentation Device of FIG. 1 with trays installed;

FIG. 12 depicts a perspective view of the Fermentation Device of FIG. 1 without any inside fixturing shown;

FIG. 13 is a block diagram depicting the major electrical components of the Fermentation Device;

FIG. 14 is a flowchart depicting a method of the present invention;

FIG. 15 is a flowchart depicting a sterilization sequence of the present invention;

FIG. 16 is a diagrammatic representation of a system of the present invention;

FIG. 17 is a diagrammatic representation of a further system of the present invention;

FIG. 18 depicts a system and related method of the present invention;

FIG. 19 depicts various inputs to the improved fermentation process of the present invention;

FIG. 20 depicts a perspective view of a further embodiment of the Fermentation Device of the present invention;

FIG. 21 depicts a front plan view of the Fermentation Device of FIG. 1;

FIG. 22 depicts a side plan view of the Fermentation Device of FIG. 21:

FIG. 23 depicts a rear plan view of the Fermentation Device of FIG. 21;

FIG. 24 depicts an alternate side plan view of the Fermentation Device of FIG. 21;

FIG. 25 depicts a top plan view of the Fermentation Device of FIG. 21;

FIG. 26 depicts a bottom plan view of the Fermentation Device of FIG. 21;

FIG. 27 depicts a perspective view of the Fermentation Device of FIG. 21 with the door open; and

FIG. 28 depicts a perspective view of the Fermentation Device of FIG. 21 with fermentation trays extended.

The present invention will be described in connection with a preferred embodiment; however, it will be understood that there is no intent to limit the invention to the embodiment described. On the contrary, the intent is to cover all alternatives, modifications, and equivalents as may be included within the spirit and scope of the invention as defined by this specification and drawings attached hereto.

### BEST MODE FOR CARRYING OUT THE INVENTION

A device that provides for fermentation of food related items is disclosed. While there are kitchen devices that maintain heat at a steady temperature, the all-in-one fermentation device as described and depicted herein provides uniform temperature control, ventilation, high relative humidity, oxygenation, and a sterilization process, in addition to maintaining a programmed temperature or temperature profile and providing software control of process and product variables. These various sub-components placed within one device provide optimal environmental conditions for different fermentation processes used in promoting health and the culinary arts and have, heretofore, not been disclosed or suggested.

The all-in-one kitchen fermentation device of the present invention accommodates sufficient volume to support different fermentation processes synchronously or at different time periods. In one embodiment of the present invention, the device will be cube-like and measure in the range of 17×17×21 inches. In further embodiments, various sizes, shapes, and number of chambers, will be employed.

The outer housing contains a handle such as a built-in-handle on the opposing sides of the device that allow for easy transport of the device. In one embodiment, the device will contain a single compartment with a single door that opens to the side, as will be further described herein. In another embodiment, two completely isolated parallel compartments that will reside side by side within the outer housing. In one embodiment, each of the two parallel compartments can be further divided into half by a removable divider (essentially quarter compartments), allowing the device to accommodate four different fermentation environments at any one time.

In one embodiment, the two main compartments will be opened and closed by two pairs of hinged double doors, each on opposing sides of the device, which allows the two compartments to be easily accessed and cleaned. In one embodiment, the roof of the device and center divider between the two internal compartments contains some of the built-in functions of the device, including temperature control elements, a humidifier, a fan, and an air pump, as will be further described below. The roof or side may, in some embodiments, house the humidifier, fan, and air pump, with the center divider containing the water supply container for the humidifier.

The following describe some examples of fermentation processes that are supported by the present invention. These are examples and are not to be considered limitations of the present invention, as the programmable and modular nature of the fermentation device of the present invention will support a wide range of fermentation processes both currently known and currently unknown.

Lactic acid fermentation is one of the simpler fermentation processes, requiring relatively anaerobic conditions and a constant temperature, ideally at 82° F., which optimizes the conditions for lactobacillus bacteria to flourish. Lactic acid fermentation can also occur at cooler temperatures, such as at ambient temperature, though the fermentation processes are slower and may allow other bacteria and molds to flourish. Lactic acid fermentation will therefore require a vessel that can provide anaerobic conditions, such as a vacuum sealed bag, a container with a vacuum-sealable lid, a container containing a salted water bath, and temperature control elements that provide a constant temperature.

Temperature control element(s) may be placed along the floor, walls and possibly roof of the unit. The temperature control element(s) are configurable so that each sub-compartment can be set at the same or at different temperatures. Customizing the temperature of each compartment allows more than one fermentation process each with different temperature requirements to occur at any one time. The temperature control elements are turned on and off by a temperature monitoring and control device that keeps the temperature within each compartment at a pre-set level. The temperature monitoring and control device communicates with the control panel on the device as well as with a mobile device application through a network such as a local Wi-Fi and Bluetooth network, which allows a user to program and set the temperature and duration of the set temperature of each compartment. Basic thin wall insulation provides better retention of heat and reduces electricity requirements.

Unlike lactic acid fermentation, which is anaerobic, certain fermentation processes such as acetic acid fermentation require oxygen, which can be achieved by directly aerating the fermentation bath. To accommodate aerobic fermentation processes, a small air pump is installed in the roof, center divider, or side, with attachable tubing that can exit either from the roof, center divider or side. In one embodiment, the tubing connects to an air filter for sanitation purposes before proceeding to a three-pronged connecting device, which allows for additional tubing to connect inferiorly to an air stone or air wand that can then be threaded into a jar through a sealable top such as a rubber stopper. The third prong can either be capped or connected to additional sequential tubing that can proceed to a second three-pronged connecting device, which would then allow for additional adjacent jars to be aerated. In one embodiment, the tubing can be sterilized by the UVC light. In another embodiment, the air pump located within the roof or center divider of the device will directly supply air via tubing to each sub-compartment. After proceeding through an air filter, separate tubing connects to each sub-compartment. Within each sub-compartment, the air pump tubing can be capped or shut off with a valve and connected to additional tubing that proceeds to an air stone or air wand, which can be threaded into the jar through a sealable top as described above. Similar to the temperature control elements, the air pump is operated by the control panel on the device and also, in one embodiment, communicates with a mobile device application through a local Wi-Fi network, which allows one to remotely program and manually turn on and off the air pump.

Koji fermentation of various types of grains, including rice, barley, or soybean, can occur in wooden or steel trays, or other type of container/vessel. Horizontal tracks or pins built into the walls of the two compartments allow the trays to be placed within the compartments. More than one tray may also be used, with each tray sliding into preconfigured tracks on top of one another. The trays will not be immediately stacked upon one another as air will still need to circulate freely throughout.

A small ultrasonic humidifier component is placed within the roof, side, and/or center divider to provide higher humidity environments for fermentation processes such as koji fermentation. The humidifier will empty the generated mist directly into the compartments through small openings in the roof, side, or center divider. Similar to the temperature control elements, the humidifier is turned on and off by a humidity monitoring and control device that maintains the humidity in the device at a pre-set level. The humidity monitoring and control device communicates with the control panel as well as with a mobile device application through a network such as, but not limited to, a local Wi-Fi network, which allows a user to program and set the humidity and duration of the desired humidity for the chamber. An adjacent removable water tank within the side, roof, floor, or main center divider provides water for the humidifier component.

Higher humidity will create pooling of water at the bottom of the chamber, so a removable metal grate within the floor will allow the water to drain into a tray underneath the floor of the device. The tray below the floor of the device will collect the excess water and can be manually removed by sliding it out and emptied. Alternatively, a small circular twist-off cap is placed within the side of the tray to allow for a tube attachment to empty the water directly into an adjacent sink.

Maintaining sufficient air circulation may be needed for ventilation of heat and humidity and is favorable for certain fermentation processes such as koji fermentation. In one embodiment, air circulation may be achieved by creating two slidable screen doors in the roof and floor that will allow for increased ventilation within the chamber. In one embodiment, a small electric powered fan may be placed in the roof, side, or floor to allow for air circulation. The electronics of the fan will communicate with the device control panel as well as with a mobile device application through a network such as a local Wi-Fi network, which will allow one to program as well as manually turn the fan on and off.

Specific to yogurt fermentation, glass jars built to specification will be provided and are stackable to maximize space efficiency within the chamber. Regular glass Mason jars or other containers of various sizes could also be used, although these jars would be less space efficient. Stackable jars built to specification are also provided in some embodiments to maximize space efficiency and accommodate different types of fermentation processes.

A method for sterilization is also provided to minimize the growth of unwanted microorganisms from and to reduce cross-contamination of fermentation products. Germicidal ultraviolet (UV) lighting may provide the easiest method of sterilization of the appliance surfaces as well pre-cleaned empty jars and fermentation vessels and air tubing from the air pump. Short wave UV-C lighting is the most effective in disinfecting the air and surfaces, which includes wavelengths of 200-280 nm, and can kill bacteria and mold and inactivate viruses, including SARS-COV-2.

Linear UV-C filaments may be placed in all compartments to ensure adequate UV exposure of the entire chamber. As a safety precaution, the UV-C lights may only turn on with the door(s) of the device completely closed. The UV-C lights can be activated through the device control panel or remotely through the mobile application.

The device may house processor and components for measuring the environmental or chemical state of the fermentation processes or the products of fermentation, for the purposes of improving the efficiency, safety, consistency, and accuracy of fermentation results. Examples of these metrics include, but are not limited to, weight, pH, carbon dioxide, sugar concentration (brix), salinity, and glutamic acid. Data for these metrics may be collected automatically and/or manually and can be compiled for individual use. In addition, data for these metrics may be collected with end-user agreement from multiple fermentation devices and stored in a central cloud database. As data for various fermentation processes are sent, additional metrics can be provided by the end-user, such as to whether a particular fermentation process and recipe were deemed successful or unsuccessful, and whether the fermentation process resulted in a better tasting product than other previous trials.

As data for these metrics accumulate, the metadata base can be further analyzed, with or without the help of computer programs, such as machine learning and artificial intelligence programs. The results of this metadata base analysis could provide clues or evidence of how to improve the efficiency, safety, consistency, and accuracy of specific fermentation processes and recipes, in a way that translates the art of fermentation into science. These results could be shared with the end-users and fermentation community and also be used to improve the internal programming of the device.

The external control panel or touch screen panel will be placed on the surface of the top or side panel of the device and provide direct control of the device, activating controls such as on/off, start/pause, and sterilize. The control panel may also display and set the desired temperature, humidity, and aeration within each compartment, as well as current and collected data regarding the environmental and chemical state of the fermentation processes or the products of fermentation. While more complicated programming functions will likely be included in the control panel, these programming functions, such as setting incubation time, temperature, humidity, fermentation aeration, and ventilation for each internal fermentation compartment, will also be programmed into an accompanying mobile device application. Similar to the other components described above, such as the temperature and humidifying modules, the control panel will communicate via a network or wireless media such as Wi-Fi to the mobile device application, allowing the application device to perform the same functions as the external control panel. As such, the device will need functions such as a Wi-Fi and Bluetooth module with internal connectivity to the various components to allow for communication to the mobile device application.

The software for the control panel on the device and for the mobile device application is capable of being updated remotely through network or wireless media such as Wi-Fi. These software updates will occur periodically to improve or expand the functionality and correct any software errors or “bugs”. With the end-user agreement, the software updates may occur either manually or automatically in the background.

The fermentation device provides optimal environmental conditions in an enclosed and insulated chamber for various fermentation processes. Optimal environmental conditions favor the survival, metabolism, and fermentation processes of organisms such as lactobacillus over the growth of undesirable bacteria, yeast, fungus, or mold. The diverse organisms involved in the various fermentation processes have different requirements for undergoing metabolism to achieve the desired fermentation process. These needs may entail an insulated environment with a constant and steady temperature that remains above room temperature for periods of time, an oxygenated environment, a relatively un-oxygenated or anaerobic environment, an environment with adequate ventilation, and/or an environment with a high relative humidity. The all-in-one fermentation device of the present invention provides multiple components capable of establishing a specific optimal condition(s).

The fermentation device provides for setting and maintaining a steady temperature and humidity within the chamber for any length of time. For fermentation processes that require oxygen, an air pump device can deliver aeration of the fermentation bath through an air wand or stone connected by tubing. Some fermentation processes require adequate ventilation within the chamber, which can be achieved with built-in sliding screen doors or a fan. Other fermentation processes require a high relative humidity that can be achieved with an ultrasonic humidifier. Internal UV-C light filaments will be installed to sterilize the chamber by delivering germicidal light radiation, killing unwanted organisms that might cross-contaminate the desired fermentation process. The device will be controlled by an external touch screen panel or control buttons as well as a mobile device application, which will require Wi-Fi and Bluetooth capabilities within the device.

The fermentation device may be made from materials such as, but not limited to, plastics, wood, glass, or stainless steel. Examples of suitable plastics include melamine, polypropylene, polyvinyl chloride, polytetrafluoroethylene, silicone, other high temperature materials, and the like. Bioplastics may also be used in some embodiments of the present invention. In addition, reinforced plastics, metals, and other materials that may be suitably formed may also be used. The fermentation device may be made by injection molding, blow molding, machining, cutting, fastening, or the like. In some embodiments, trays may be made from wood, glass, stainless, steel, plastic, or the like. In some embodiments, glass jars or vessels may be employed within the Fermentation Device. Electrical, mechanical, and electro-mechanical components may also be added to the Fermentation Device as described and depicted herein.

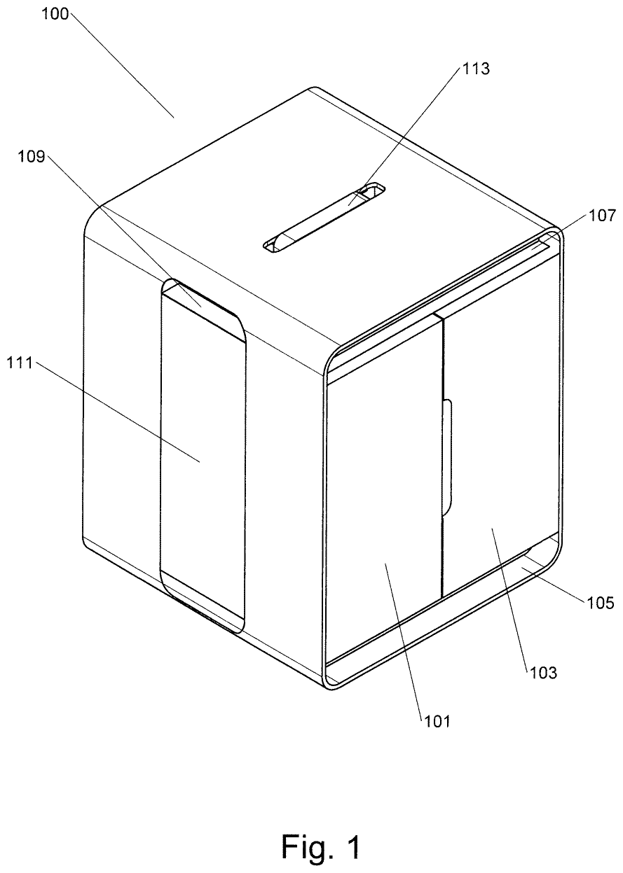

FIG. 1 depicts a perspective view of one embodiment of the Fermentation Device of the present invention. The Fermentation Device 100 comprises two separate fermentation compartments, with access to each compartment by way of a door or doors. In one embodiment, a first door 101 and a second door 103 can be seen hinged to or otherwise fastened to the fermentation device body. The doors are gasketed to ensure proper and sufficient sealing. A drainage tray 105 is fitted below each compartment to allow for the collection and removal of water from humidification or other liquids from the fermentation process. Toward the top of each compartment is a ventilation opening 107 to allow for the ventilation of each chamber. The ventilation opening 107 may employ a screen, filter, or other element to ensure that no contaminants enter the fermentation chamber. On each side of the fermentation device 101 is a handle 109 that may, in some embodiments, be recessed. A control panel 111 may also be fastened or built with a side. The control panel may contain switches and dials, or may, in some embodiments, be a touch screen or similar user interface device (UID). A humidifier 113 such as an ultrasonic humidifier can be seen as well where the humidifier output is directed to one or both fermentation chambers, and can be controlled by way of the control panel 111.

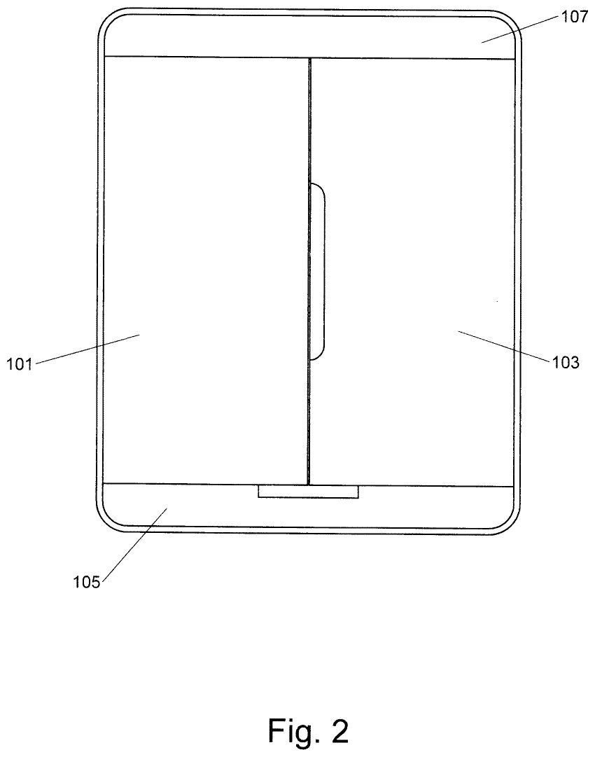

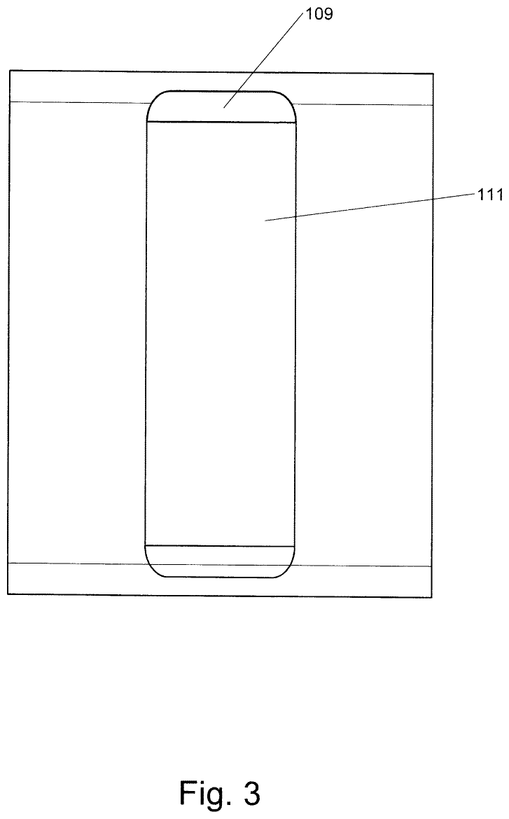

FIG. 2 depicts a front plan view of the Fermentation Device 100. In this view, the double doors 101 and 103 can be seen along with a handle or recess to facilitate opening and closing of the doors. The ventilation opening 107 can be seen along with the drainage tray area 105. Paired fans for ventilating the compartments, such as during koji fermentation, are located within the top panel of the device. Ventilation screens along the upper side panels can assist in ventilating the compartments. Each fan is associated with a fermentation compartment, and may be controlled individually through the control panel 111 (not shown in FIG. 2, see FIGS. 1 and 3).

FIG. 3 depicts a side plan view of the Fermentation Device 100 showing the first handle 109 and the control panel 111. A second handle is located on the opposite side of the Fermentation Device (not shown).

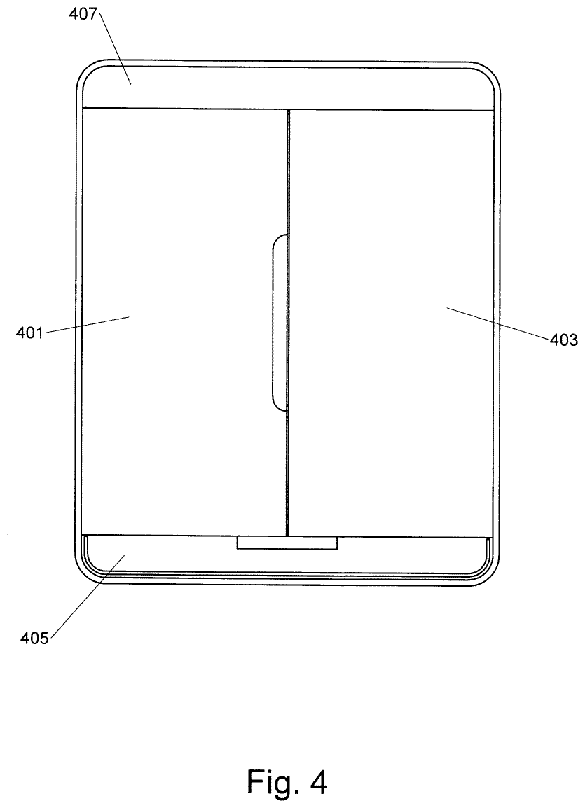

FIG. 4 depicts a rear plan view of the Fermentation Device 100. As previously described, the Fermentation Device comprises a first fermentation compartment and a second fermentation compartment. Each fermentation compartment is separate from the other, and is accessed by doors such as the double doors 401 and 403 shown in FIG. 4. The rear of the device also has a drainage tray 405 and a ventilation opening 407.

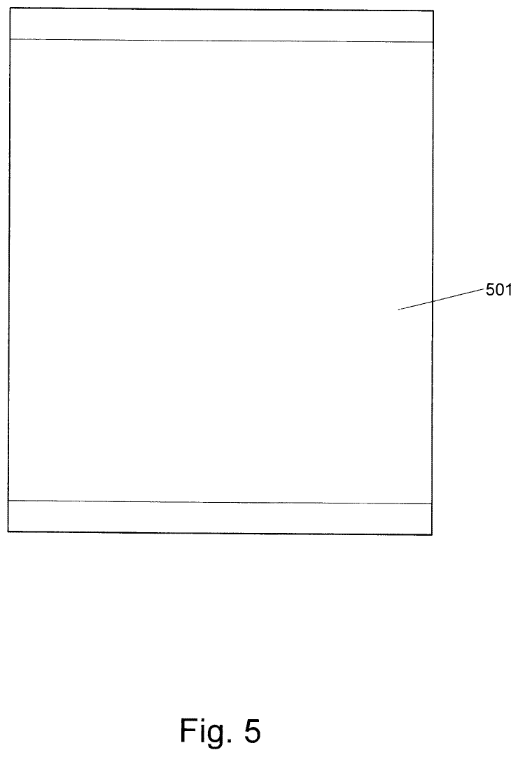

FIG. 5 depicts an alternate side plan view of the Fermentation Device 100 showing the side 501.

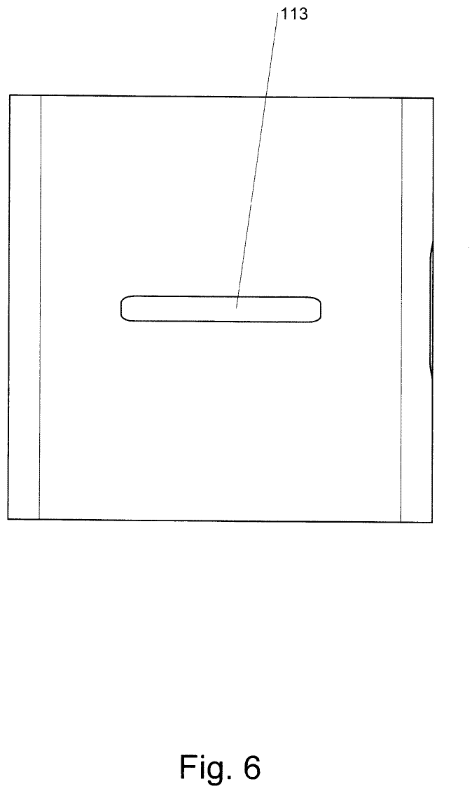

FIG. 6 depicts a top plan view of the Fermentation Device 100 showing the humidifier 113 inserted or otherwise installed in the top of the fermentation device.

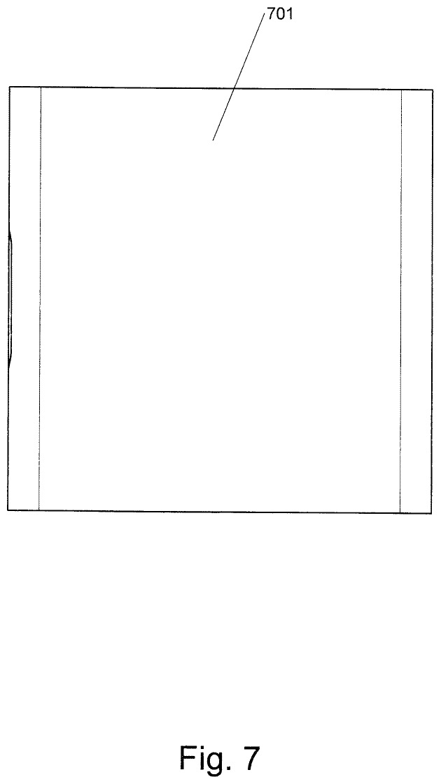

FIG. 7 depicts a bottom plan view of the Fermentation Device 100 showing the bottom of the fermentation device 701.

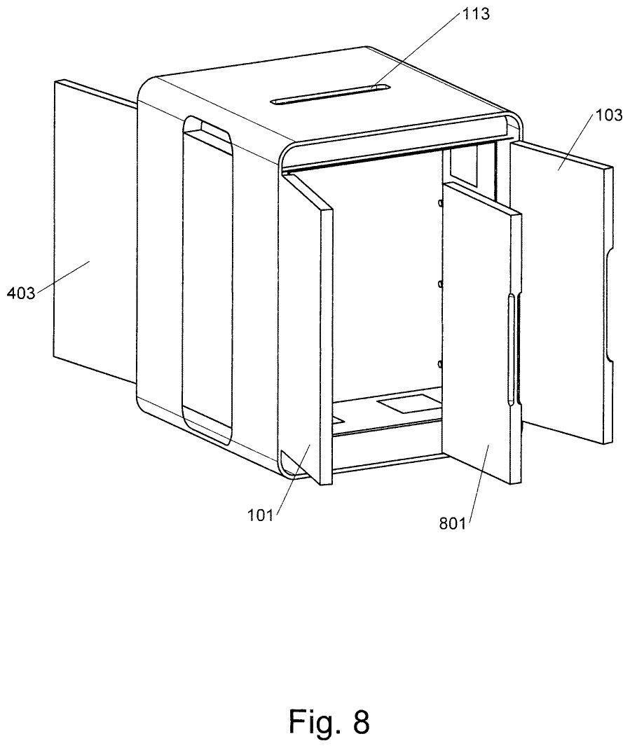

FIG. 8 depicts a perspective view of the Fermentation Device 100 with the doors open and a removable partition 801 depicted in free space and not yet installed. The doors in both fermentation chambers are open in this view. It should be noted that the removable partition 801 may be placed in a variety of positions to create at least two additional fermentation chambers, albeit smaller.

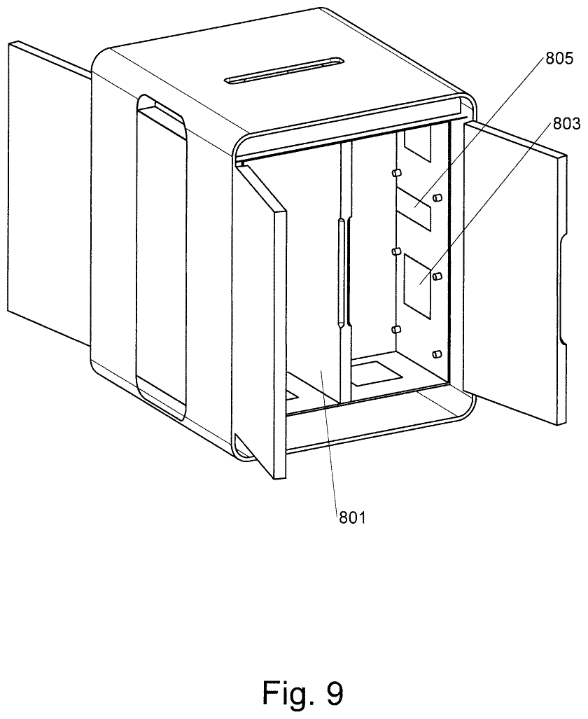

FIG. 9 depicts a perspective view of the Fermentation Device 100 with the removable partition 801 of FIG. 8 installed. An ultraviolet sterilizer 803 as well as a heating unit 805 can be seen.

Temperature control units are placed along the side panel and floor of one of the two compartments. The trays have been removed for better visualization. The temperature control elements or units are grouped spatially, so that the temperature control elements contributing to the thermal management of one quarter or half chamber can be set to a different temperature than an adjacent chamber. Each group of temperature control elements can be synchronously turned on and off by either the external control panel touch pad or by a mobile device application, which will require Wi-Fi connectivity. The various groups of temperature control elements can also be synchronously turned on and off, allowing one to easily set the entire chamber to one temperature. Temperature control elements include, but are not limited to, heating elements such as inductive or resistive heating devices, as well as cooling devices that are compressor based or may be dual mode such as Peltier devices and other thermal control modules.

An ultraviolet sterilizer UV-C light is installed into the far-right side panel. The UV-C lights provide a means of safely irradiating the field for sterilization purposes and will only turn on with the doors completely closed. A safety interlock switch or sensor such as an optical or magnetic is operably connected with the doors. In some embodiments of the present invention, additional UV-C lamps will be placed within the fermentation chambers to facilitate adequate sterilization.

Above the humidifier within the midline center divider is an air pump (not shown) that can be attached to tubing within either compartment. The air pump allows aeration of a fermentation bath, such as with acetic acid fermentation, using an air stone or air wand for example.

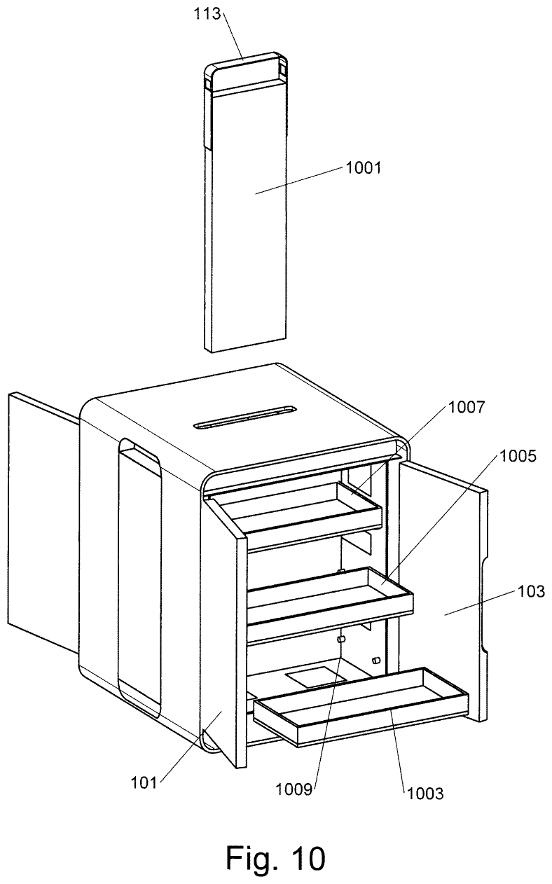

FIG. 10 depicts a perspective view of the Fermentation Device 100 configured with trays installed. In some embodiments of the present invention, the trays are made from wood. Shown removed from the Fermentation Device 100 is a humidifier 113 such as an ultrasonic humidifier. A water tank 1001 can be seen attached to the humidifier 113. Stackable fermentation trays 1003, 1005 and 1007 can be seen. In some embodiments, additional or otherwise configured trays may be employed.

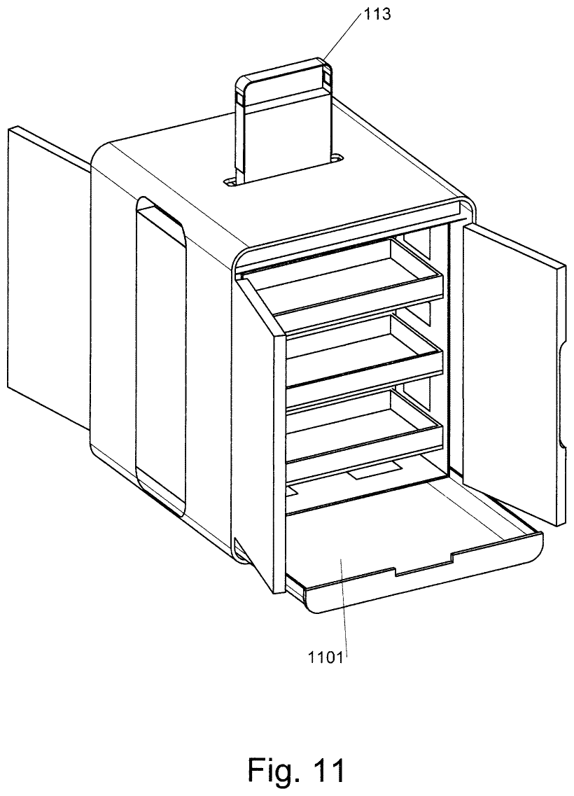

FIG. 11 depicts a perspective view of the Fermentation Device 100 with trays installed fully and the drainage tray 1101 shown in an extended position.

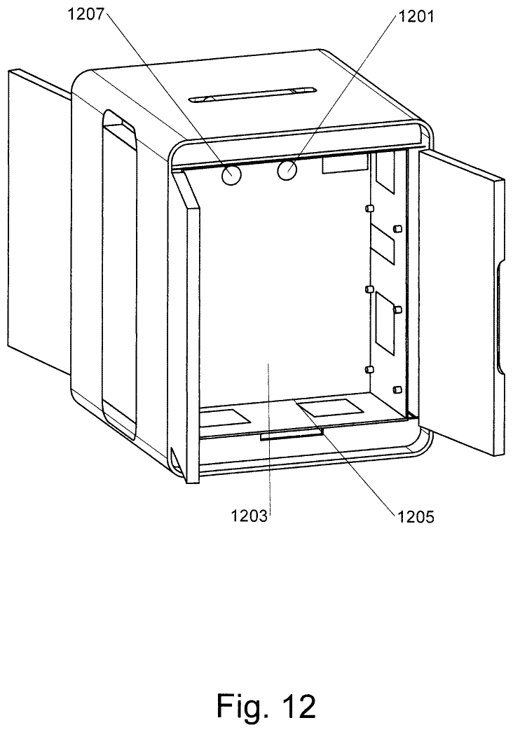

FIG. 12 depicts a perspective view of the Fermentation Device 100 without any inside fixturing shown. An air mister 1201 and 1207 can be seen toward the back of the fermentation chamber depicted. The air mister provides moisture to the fermentation chamber and can be controlled through the control panel. Each air mister is placed such that each chamber formed from placement of the movable partition has an air mister that can be independently controlled. A drain 1205 at the back of each fermentation chamber allows water or other liquid to drain from each fermentation chamber into the drainage tray 1101.

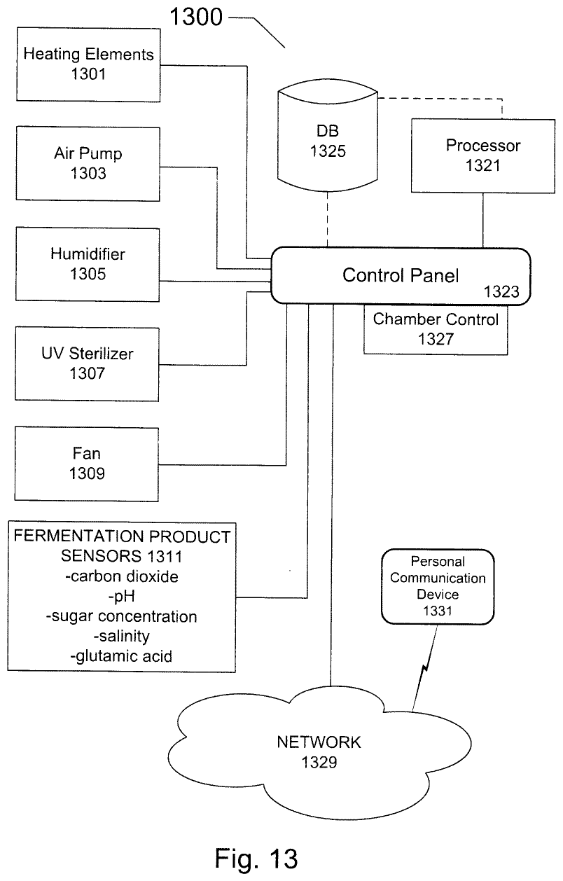

FIG. 13 is a block diagram 1300 depicting the major electrical components of the Fermentation Device. As described previously, a control panel 1323 is located on the fermentation device and may, in some embodiments, be a touch screen. The control panel 1323 in some embodiments has an interface to a cellular or wi-fi network 1329 to allow connectivity by a personal communications device 1331 such as, but not limited to, a smart phone, Personal communications devices include, but are not limited to, phones, tablets, glasses, virtual headsets, watches, and the like.

In some embodiments, the wireless connection between the smart phone and the control panel 1323 is Bluetooth. The control panel 1323 may also contain chamber control 1327 where the user can specify fermentation process parameters that are unique to a specific fermentation chamber in the fermentation device. The control panel 1323 may be connected with a processor 1321 that contains software and control logic for the operation of the fermentation device. The processor 1321 may optionally be connected with a database (DB) 1325 that contains process maps (“recipes” or process related parameters that are unique to the fermentation process being programmed into the Fermentation Device.

The control panel 1323 controls the various process related devices contained in the Fermentation Device. Temperature Control Elements 1301 can be programmed for a specific temperature and run time. Temperature profiles may also be defined through the control panel 1323. Each fermentation chamber may have a unique temperature control element or temperature control elements such that temperatures can be defined differently across the various fermentation chambers within the fermentation device as configured. An Air Pump 1303 can be activated and controlled through the control panel 1323 such that air is provided through a tube or series of tubes into the fermentation product for specific fermentation processes. A humidifier 1305 such as an ultrasonic humidifier can be activated and controlled by the control panel 1323 to provide a specified humidity to a fermentation chamber. An Ultraviolet (UV) Sterilizer 1307 or plurality of Ultraviolet Sterilizers may also be activated and controlled from the control panel 1323. In addition, a fan 1309 or multiple fans can be activated and controlled from the control panel to control airflow into and out of each fermentation chamber. To ensure that the programmed process parameters are maintained throughout the specified fermentation process, sensors 1311 are incorporated into the fermentation device. Sensors include temperature sensors, humidity sensors, light/UV-C sensors, airflow sensors, and a scale to measure the weight of the fermentation vessel. Additional sensors may also be employed to detect various process products or bi-products such as carbon dioxide, pH, sugar concentration, salinity, glutamic acid, and the like.

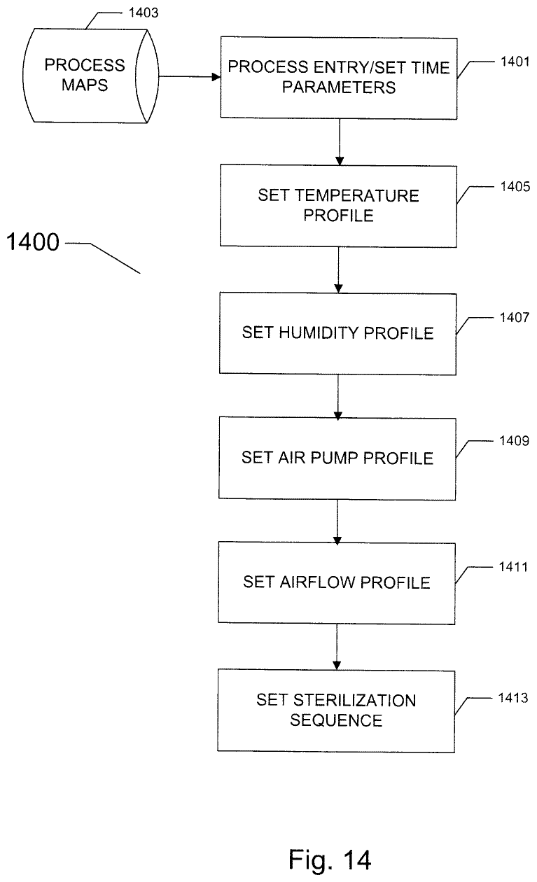

FIG. 14 is a flowchart 1400 depicting a method of the present invention 1400 where a user begins a desired fermentation process through control panel input. The user has the ability to manually set each process parameter or to draw from process maps stored in a database 1403 where the process maps define various fermentation processes such that the user can pick from a desired fermentation process without the need to program the process parameters. Additionally, the user may add their own process maps such that repeat fermentation processes become extremely automated and easy to initiate. It should be noted that each process parameter can be specified for a unique fermentation chamber, as the fermentation device of the present invention contains individual fermentation chambers that can be configured by the user. While FIG. 14 depicts sequential steps, the steps may be performed in any order, and are not limited to any particular sequence. In addition, steps may be omitted, or additional steps added in some embodiments of the present invention.

In step 1401 the user begins either a manual process entry or executes a stored process map, which can occur at the present or future time. Should the user elect to manually enter the process parameters, the fermentation time is specified in step 1401. In step 1405 the temperature profile is set where the temperature can be specified throughout the fermentation process. In step 1407, a humidity profile is set up where the humidity can be specified throughout the fermentation process. In step 1409, if an air pump is used to sparge the fermentation product, an air pump profile is specified where airflow as well as time are specified. In step 1411 an airflow profile is specified where fans are used to move air through the specified fermentation chamber and the rate of airflow as well as the time duration of the specified airflow is entered. Lastly, in step 1413 a sterilization sequence is specified where the intensity and duration of UV light exposure is defined. In some embodiments, there may be more than one UV light per fermentation chamber. Once the process parameters are specified either manually or through the use of a process map, the fermentation process begins through automated execution of the defined process profiles. Sensors, as previously described, monitor and adjust the ongoing fermentation process to ensure that the defined process parameters are followed.

Sterilization is a critical part of producing fermentation products, as contamination from pathogens is a serious health concern. As such, the present invention provides for UV-C disinfection of the device. It is important to provide sterilization of the device only when the fermentation product is not present, as this would otherwise inactivate the fermentation product upon exposure to the UV-C sterilization light. As such, a series of software based steps are executed on a processor having memory and access to computer readable media.

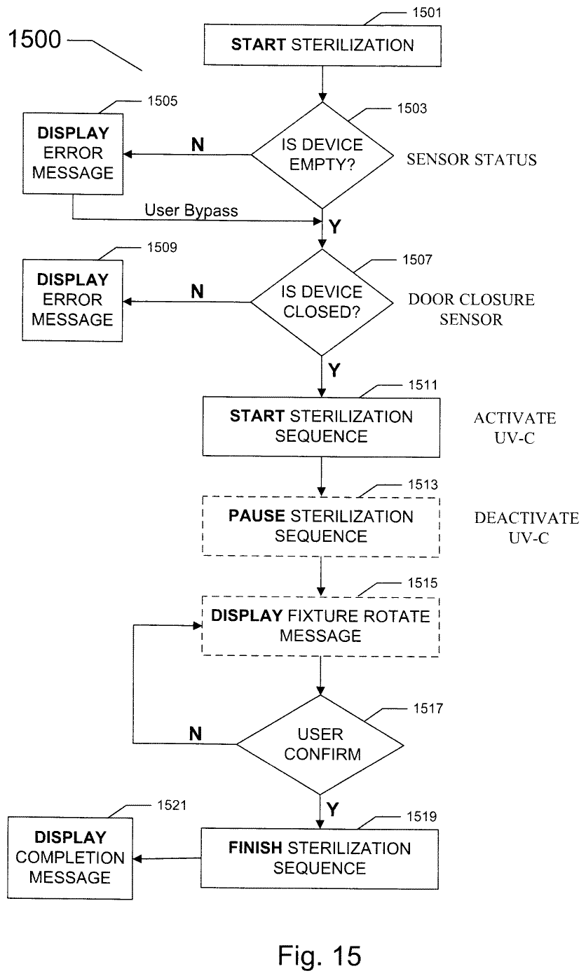

FIG. 15 is a flowchart 1500 depicting a sterilization sequence of the present invention. The user starts a sterilization process after fermentation is complete and before starting another fermentation cycle by commencing a start sterilization command in step 1501 through entry of such a command at the control panel or a remote device of the present invention. Such a remote device may be, for example, a smart phone, a portable computer, or the like. A sensor or interlock such as a load cell or optical sensor provides a status to indicate if the fermentation device is empty in step 1503. The interlock may comprise a weight sensor configured to detect weight of a fermentation product or fixture in the chamber. If the fermentation device is not empty as evident by the status of the sensor, an error message is displayed to the user in step 1505, again either at the control panel or through a remote device. Should the user desire to sterilize a cleaned vessel prior to its use for a fermentation process, the user will be able to override or bypass the error message and confirm that the user indeed would like the chamber and contained vessel sterilized. If the device is empty as evident by the status of the sensor, the software determines if the fermentation device is closed through a door closure sensor, interlock or similar sensing arrangement in step 1507. If the fermentation device door is not closed, an error message is displayed to the user in step 1509, again either at the control panel or through a remote device. If the door is closed, the sterilization sequence is started in step 1511 through activation of the UV-C element(s). In some embodiments, after a specified sterilization time the sterilization sequence is paused in step 1513 and the UV-C element(s) are deactivated, a fixture rotate message is displayed to the user in step 1515 and the user then rotates or otherwise reconfigures the interior of the fermentation device to allow for proper irradiation of all interior areas of the fermentation device. The sterilization sequence will remain paused until such time as the user confirms that the rotation has been performed in step 1517, upon which time the sterilization sequence is completed through another activation of the UV-C element(s) in step 1519 and a completion message is displayed to the user in step 1521. It should be noted that the duration and intensity of UV-C exposure is specified based on the internal device configuration as well as the fermentation process previously employed. While these sterilization steps provide for a sterile fermentation device, it should be noted that the sterilization steps may, in some embodiments, simply be an activation and deactivation of a UV-C sterilization element or elements or may employ other sterilization means such as, for example, chemical or heat based sterilization.

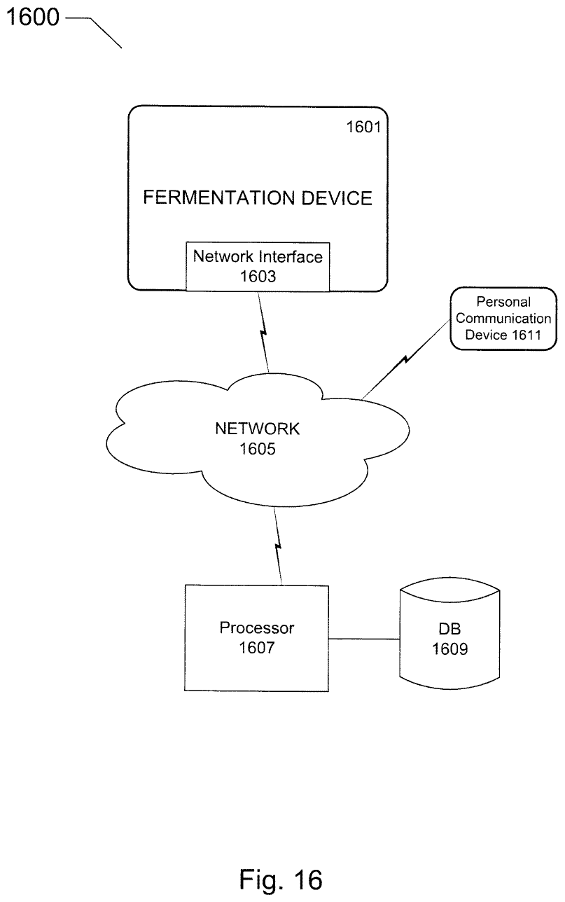

The fermentation device of the present invention produces improved fermentation products through not only the all in one nature of the fermentation device, but also in the use of process parameters that are stored and available to a user either locally (in the fermentation device itself) or through a system 1600 such as depicted in FIG. 16. The fermentation device 1601 in some embodiments includes a network interface 1603 that connects with a network 1605 such as the internet. Also connected to the network 1605 is a processor 1607 that is operatively connected to a database or data structure 1609 where the database or data structure 1609 contains process parameters (recipes) for the production of various fermentation products as well as product parameters detailing the qualities of a product produced by the selected process parameter. In addition, the system depicted in FIG. 16 provides remote user access by way of a personal communication device 1611 such as, but not limited to, a smartphone or other electronic device where the user can control the fermentation device and receive updates regarding the fermentation process underway.

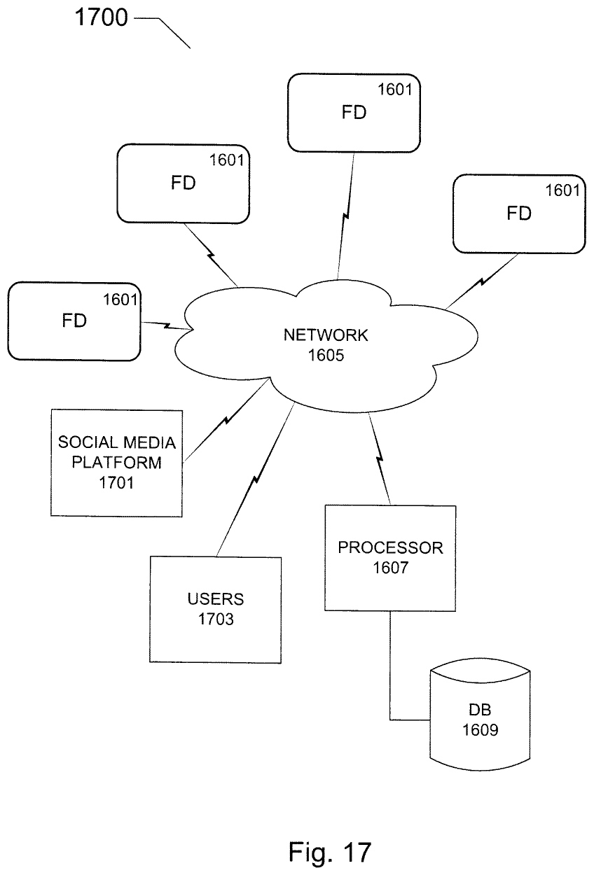

To build on the network based environment described in FIG. 16 and as depicted by the system 1700 in FIG. 17, in some embodiments multiple fermentation devices 1601 are connected to a network 1605 such as the internet that is in turn connected to a processor 1607 which is connected to or has access to a database or data store 1609 where process and product parameters are stored for user access and retrieval. In order to create improved fermentation products, an “ecosystem” of users 1703 is created where many users provide process and product parameters to the processor and database system and an exchange of information takes place on a social media platform 1701 or similar such environment. Users provide feedback, comments and interaction with other users specific to the fermentation product that they are producing with the fermentation device of the present invention. This allows for the creation of a wealth of process maps that include process and product parameters, resulting in improved fermentation products through the collective user base and artificial intelligence to bring order and structure to the many elements of user input received in such a system.

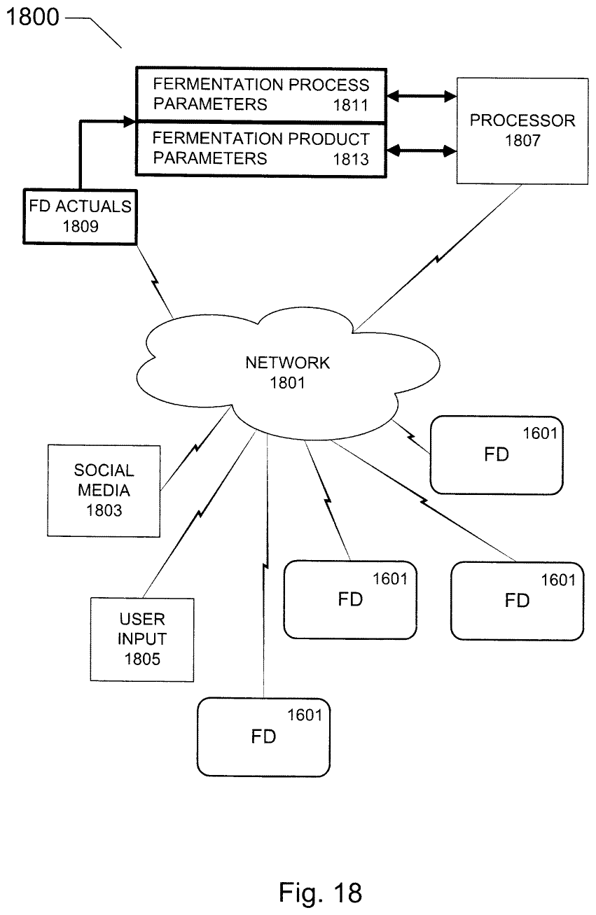

In FIG. 18, a system and related method of the present invention 1800 is depicted where a plurality of fermentation devices 1601 are connected to a network 1801 that includes access to social media 1803 or a similar platform and user input 1805 is received and stored through a processor 1807 that contains fermentation process 1811 and product 1813 parameters as previously described. The fermentation process and product parameters create a process map that defines both the fermentation process variables as well as the resulting product attributes. Such a process map can then be sent from the processor 1807 to a target fermentation device 1601 where the process map then controls the fermentation steps executed by the fermentation device. User input includes additional or modified fermentation process and product parameters as well as fermentation device actuals 1809 either sent by a user or directly sent by the fermentation device itself. Users may also post comments related to the fermentation process map or other fermentation related topics. This social networking or ecosystem results in superior fermentation products as well as variety and advancement of fermentation product creation.

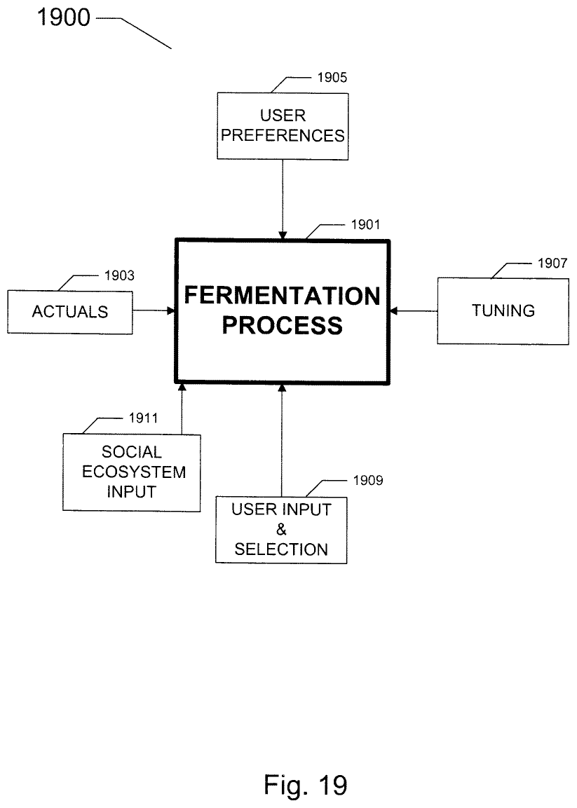

FIG. 19 depicts various inputs 1900 to the improved fermentation process 1901 of the present invention. Actual fermentation results 1903 may be provided by a user or the fermentation device itself. User preferences 1905 include, for example, sweetness, sourness, texture, consistency, and the like and are used to create modified process maps. Tuning 1907 of the process maps includes process changes to address environmental conditions such as altitude, modified or new process maps, and the like. User input and selection 1909 provides user choice and social ecosystem input 1911 includes the creation of new process maps and modification of existing process maps based on user input.

While a double door version of the fermentation device has been previously depicted by way of FIGS. 1-12, a single door version of the fermentation device is also described and depicted herein. The single door version of the fermentation device is essentially a different chamber, but the components that make up the fermentation device such as a temperature control device configured to provide heat transfer to the chamber; a ventilation fan attached to the chamber; an air pump configured to provide delivery of oxygen into a fermentation bath; a humidifier configured to provide moisture to the chamber; and an ultraviolet sterilization light source configured to irradiate the inside of the chamber remain the same. A chamber, as defined herein, includes any housing or cabinet sufficient to contain a fermentation product, regardless of the number of doors, shelves, trays, or other such fixturing.

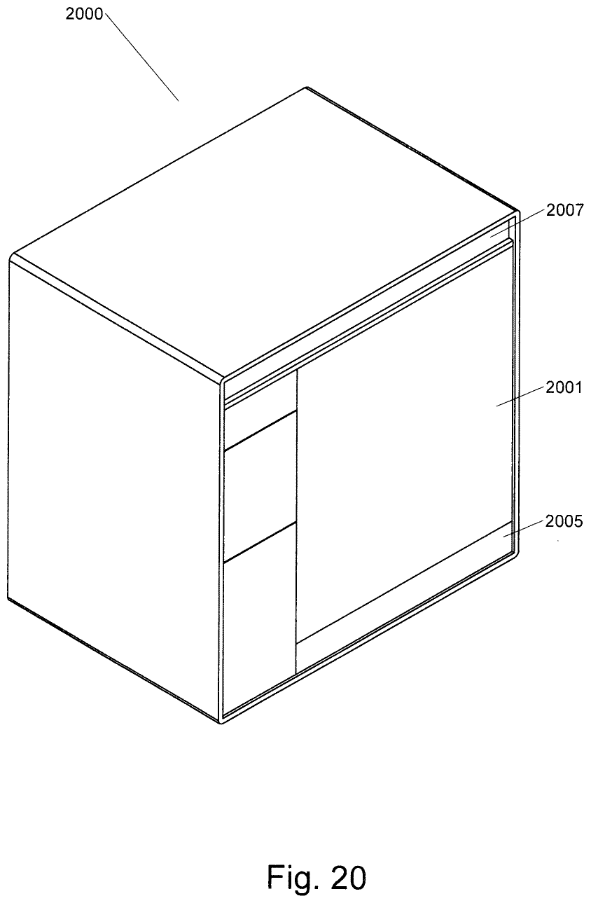

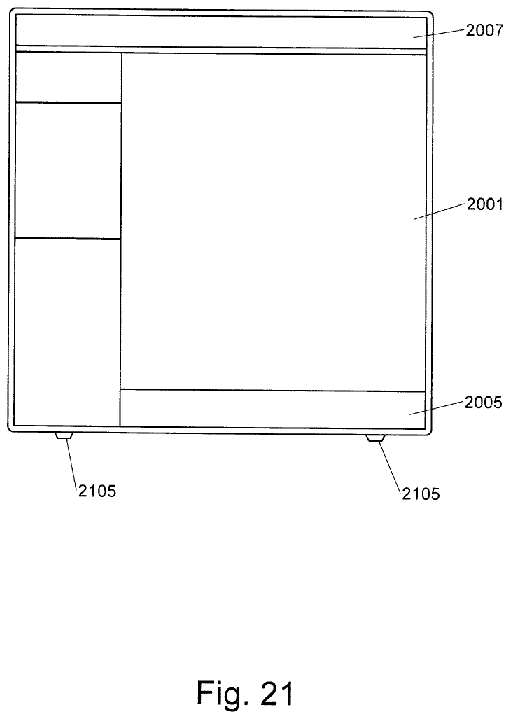

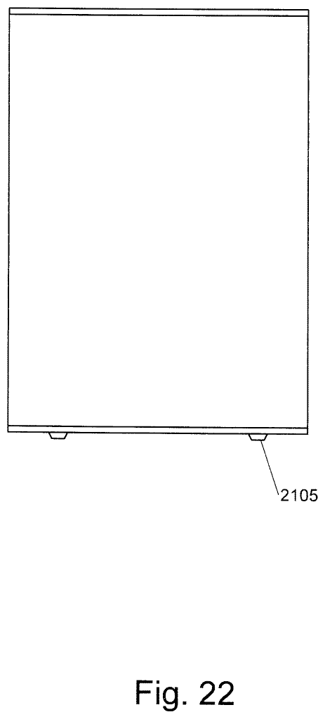

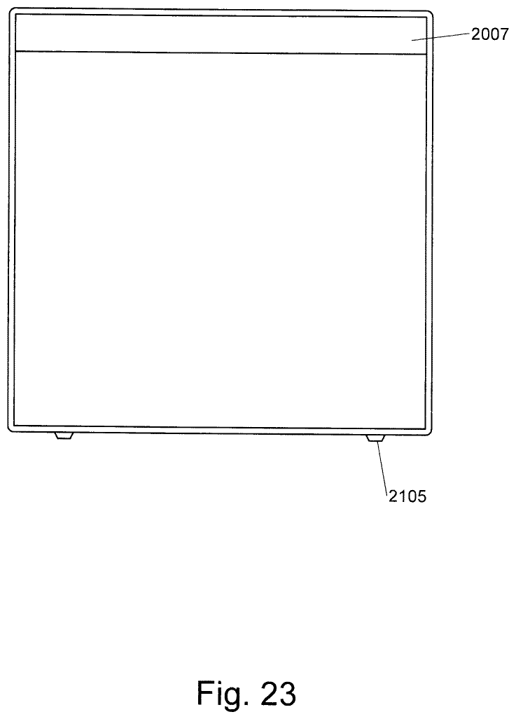

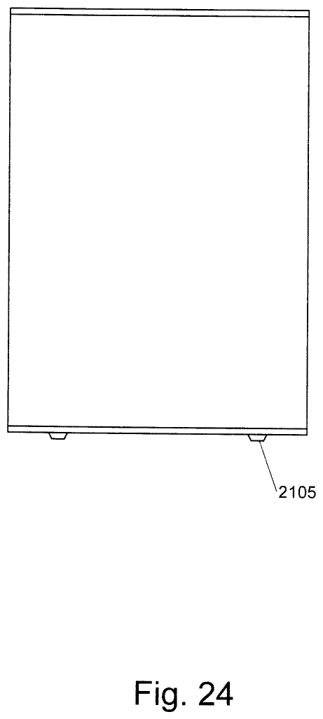

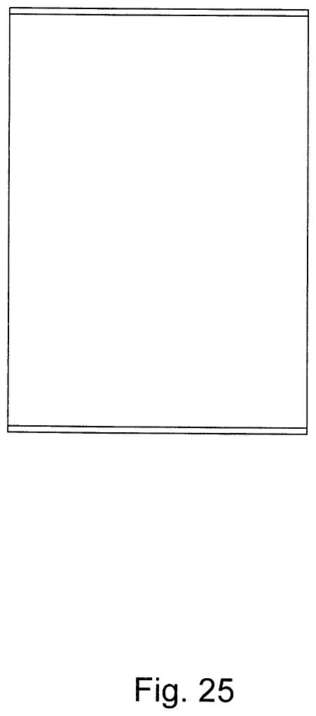

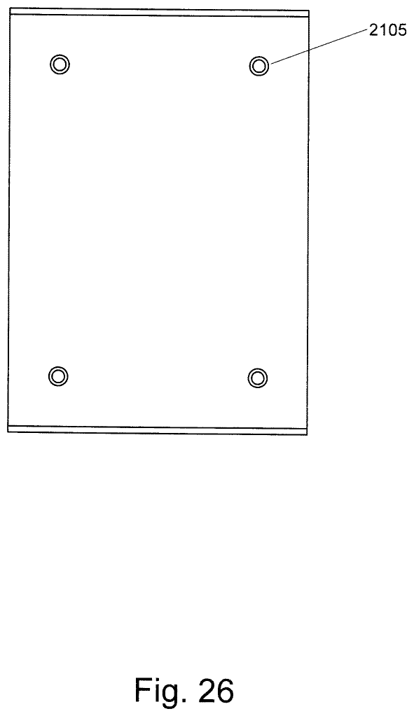

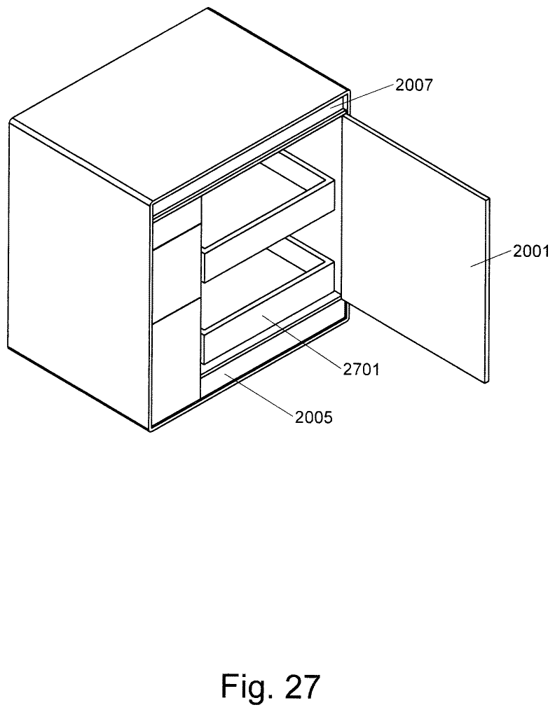

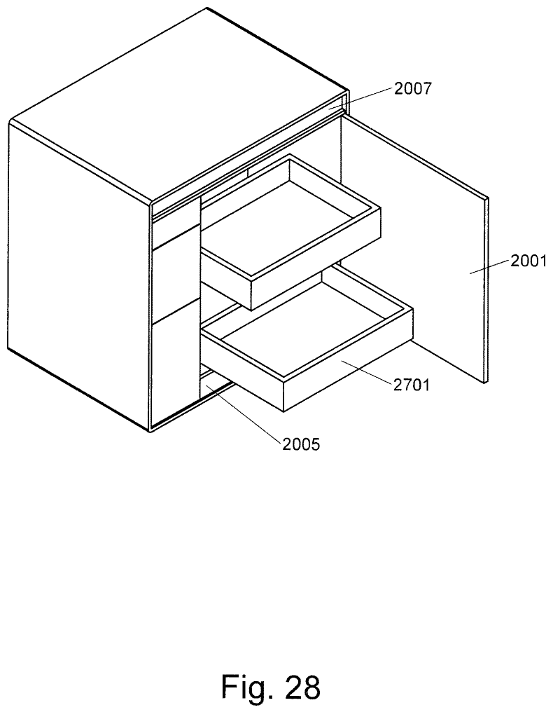

FIGS. 20-28 depict such a single door chamber. The chamber as depicted in FIGS. 20-28 is configured to include the components described herein. FIG. 20 depicts a perspective view of a further embodiment 2000 of the Fermentation Device of the present invention which employs a single door chamber. A single door 2001 is depicted with space for a control panel or panels to the side of the single door 2001. A drainage tray 2005 can be seen to the bottom of the chamber and a ventilation opening 2007 can be seen toward the top of the chamber. FIG. 21 depicts a front plan view of the Fermentation Device of FIG. 1 where feet 2105 can be seen affixed to the bottom of the fermentation device 2000. FIG. 22 depicts a side plan view of the Fermentation Device of FIG. 21. FIG. 23 depicts a rear plan view of the Fermentation Device of FIG. 21. FIG. 24 depicts an alternate side plan view of the Fermentation Device of FIG. 21. FIG. 25 depicts a top plan view of the Fermentation Device of FIG. 21. FIG. 26 depicts a bottom plan view of the Fermentation Device of FIG. 21. FIG. 27 depicts a perspective view of the Fermentation Device of FIG. 21 with the door open where a fermentation tray 2701 can be seen. Other interior fixturing such as partitions, supports, shelves, racks, bottle racks or holders, and the like may also be employed depending on the fermentation process being used. Lastly, FIG. 28 depicts a perspective view of the Fermentation Device of FIG. 21 with fermentation trays extended.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, claims and drawings appended herein.

What is claimed is:

1. A fermentation device for producing improved fermentation products comprising:
a chamber;
a temperature control device configured to provide heat transfer to the chamber;
a ventilation fan attached to the chamber;
an air pump configured to provide delivery of oxygen into a fermentation bath;
a humidifier configured to provide moisture to the chamber; and
an ultraviolet sterilization light source configured to irradiate the inside of the chamber.

2. The fermentation device of claim 1, wherein the ultraviolet sterilization light source comprises UV-C light at a wavelength of 200-280 nanometers.

3. The fermentation device of claim 1, further comprising an interlock to prevent the ultraviolet sterilization light source from turning on during an active fermentation process within the chamber.

4. The fermentation device of claim 3, wherein the interlock comprises a weight sensor configured to detect weight of a fermentation product in the chamber.

5. The fermentation device of claim 3, wherein the interlock comprises an optical sensor configured to detect the presence or absence of a fermentation product in the chamber.

6. The fermentation device of claim 1, wherein the chamber is divided into compartments where each compartment is configured to support a separate fermentation process.

7. The fermentation device of claim 1, further comprising a network interface.

8. The fermentation device of claim 1, wherein the network interface is configured to provide remote operation of the device from a personal communications device.

9. The fermentation device of claim 1, wherein the personal communications device is a smart phone.

10. The fermentation device of claim 1, wherein the network interface supports connection to a wi-fi network.

11. A system for the production of improved fermentation products, the system comprising:
The fermentation device of claim 7;
a computer having a processor, memory and access to computer readable media, the computer configured to connect to the network interface of the fermentation device;
wherein the computer is configured to connect with a data store or database containing process maps where each process map contains fermentation process and fermentation product parameters; and
wherein the computer is configured to provide a selected process map to the fermentation device.

12. The system of claim 11, wherein the fermentation device is configured to receive a provided process map and translate the provided process map into control instructions.

13. The system of claim 11, wherein the network interface of the fermentation device is configured to provide remote operation of the device from a personal communications device.

14. The system of claim 13, wherein the personal communications device is a smart phone.

15. The system of claim 11, wherein the computer is configured to receive fermentation process parameters and associated fermentation product parameters from at least one fermentation device.

16. The system of claim 15, wherein the received fermentation process and fermentation product parameters are provided to a network.

17. The system of claim 16, wherein the provided fermentation process and fermentation product parameters are shared on a social media platform.

18. The system of claim 16, wherein the provided fermentation process and fermentation product parameters are categorized to allow for the production of improved fermentation products.

19. A method for the production of improved fermentation products, the method comprising the steps of:
operating the fermentation device of claim 7;
creating fermentation process and fermentation product parameters from the operation of the fermentation device of claim 7;
storing the created fermentation process and fermentation product parameters on a computer; and
allowing access to the computer by a user.

20. The method of claim 19, further comprising the step of:
allowing the user to post comments associated with a selected fermentation process and fermentation product parameters.
